(19) United States
(12) Patent Application Publication
(10) Pub. No.: US 2022/0228128 A1
(43) Pub. Date: Jul. 21, 2022

(54) USE OF POXVIRUS WITH AUTOLOGOUS INDUCED PLURIPOTENT STEM CELLS FOR VACCINATION AND DISEASE THERAPY

(71) Applicant: IMMUNOLUX INTERNATIONAL CORP., Highland, CA (US)

(72) Inventor: Aladar SZALAY, Highland, CA (US)

(21) Appl. No.: 17/615,018

(22) PCT Filed: May 29, 2020
(86) PCT No.: PCT/US20/35103

§ 371 Date: Nov. 29, 2021

Related U.S. Application Data
(60) Provisional application No. 62/911,640, filed on Oct. 7, 2019.
(60) Provisional application No. 62/879,196, filed on Jul. 26, 2019.
(60) Provisional application No. 62/854,817, filed on May 30, 2019.

(51) Int. Cl. C12N 5/074; C12N 15/86

(52) U.S. Cl. CPC C12N 5/0696; C12N 15/86; C12N 2710/24143; C12N 2710/24132; C12N 2510/00

(57) ABSTRACT

Provided herein are compositions of induced pluripotent stem cells (iPSCs), or pancreatic beta cells, and poxvirus, and methods of making and using the same to treat disease.

### BACKGROUND

Cancer is the second most common cause of death in the United States, exceeded only by heart disease. In the United States, cancer accounts for 1 of every 4 deaths. Discovering highly effective cancer treatments is a primary goal of cancer research.

Inflammatory diseases, for example autoimmune diseases, are caused by chronic inflammation in a subject. These diseases can cause symptoms ranging from mild discomfort to severe reactions, and even death.

Infectious diseases are caused by organisms, such as bacteria, viruses, fungi or parasites. While infections are often treated with antibiotics, antivirals, antifungals, antiprotozoals, and antihelminthics, pathogens are becoming increasingly resistant to these drugs. Other pathogens have no known treatments.

New methods of treating these diseases are needed.

### SUMMARY OF THE INVENTION

The instant technology generally relates to methods and compositions for treating a disease in a subject in need thereof by administering to the subject a poxvirus and an induced pluripotent stem cell (iPSC). The instant technology also relates to methods and compositions for treating a disease in a subject in need thereof by administering to the subject a poxvirus and a pancreatic beta cell.

In one aspect a composition for treating a disease is provided. The composition may comprise a poxvirus (or other oncolytic virus) and an iPSC. In embodiments, the iPSC is derived from an ectodermal cell type. In embodiments, the iPSC is derived from an endodermal cell type. In embodiments, the iPSC is derived from a mesodermal cell type.

In embodiments, the iPSC is derived from a subject to be treated with the composition.

In one aspect, the composition comprises a poxvirus (or other oncolytic virus) and a pancreatic beta cell. In embodiments, the beta cell is derived from a stem cell. In embodiments, the beta cell is derived from an iPSC.

In embodiments, the poxvirus is a vaccinia virus. In embodiments, the vaccinia virus is a smallpox vaccine. In embodiments, the vaccinia virus is selected from Dryvax, ACAM1000, ACAM2000, Lister, EM63, LIVP, Tian Tan, Copenhagen, Western Reserve, Modified Vaccinia Ankara (MVA), New York City Board of Health, Dairen, Ikeda, LC16M8, Western Reserve Copenhagen, Tashkent, Tian Tan, Wyeth, IHD-J, and IHD-W, Brighton, Dairen I and Connaught strains. In embodiments, the vaccinia virus is ACAM1000. In embodiments, the vaccinia virus is ACAM2000. In embodiments, the vaccinia virus is a New York City Board of Health strain.

In embodiments, the poxvirus is an attenuated virus.

In embodiments, the stem cell includes a recombinant polynucleotide that encodes a therapeutic molecule. In embodiments, the beta cell includes a recombinant polynucleotide that encodes a therapeutic molecule. In embodiments, the poxvirus includes a recombinant polynucleotide that encodes a therapeutic molecule. In embodiments, the therapeutic molecule is a molecule that treats a disease or a symptom of a disease (e.g., a therapeutic antibody or antibody fragment, such as an anti-cancer antibody or fragment, or a therapeutic antibody or antibody fragment that treats an inflammatory disease; a therapeutic fusion protein, such as an anti-cancer or anti-inflammatory disease fusion protein; an antibiotic; a toxin; a cytokine; an enzyme, etc.).

In embodiments, the composition also includes chimeric antigen receptor (CAR)-T cell. In embodiments, the CAR-T cell and the iPSC were derived from the same individual. In embodiments, the CAR-T cell and the beta cell were derived from the same individual. In embodiments, the CAR-T cell and/or the iPSC were derived from a patient to be treated with the therapeutic composition. In embodiments, the CAR-T cell and/or the beta cell were derived from a patient to be treated with the therapeutic composition. In embodiments, the CAR-T cell and the iPSC were derived from different individuals. In embodiments, the CAR-T cell and the beta cell were derived from different individuals. In embodiments, the CAR targets an antigen associated with a disease. In embodiments, the disease is cancer, an inflammatory disease, or an infectious disease.

In one aspect a method for treating a disease in a subject is provided. The method may comprise administering a poxvirus (or other oncolytic virus) and an iPSC to the subject. The method may comprise administering a poxvirus (or other oncolytic virus) and a beta cell to the subject. In embodiments, the disease is a cancer or tumor. In embodiments, the disease is an infectious disease. In embodiments, the disease is an inflammatory disease. In embodiments, the disease is an autoimmune disease. In embodiments, the disease is characterized by chronic inflammation in the subject.

In embodiments, the chronic inflammatory disease is selected from asthma, chronic peptic ulcer, tuberculosis, arthritis, periodontitis, ulcerative colitis, Crohn's disease, sinusitis, active hepatitis, atherosclerosis, dermatitis, inflammatory bowel disease (IBS), systemic lupus, fibromyalgia, Type 1 diabetes, psoriasis, Multiple sclerosis, Addison's disease, Grave's disease, Sjogren's syndrome, Hashimoto's thyroiditis, Myasthenia gravis, vasculitis, pernicious anemia, or celiac disease.

In embodiments, a CAR-T cell is also administered to the subject. In embodiments, the CAR targets an antigen associated with the disease. In embodiments, the CAR-T cell is autologous. In embodiments, the CAR-T cell is allogeneic.

In embodiments, a therapeutic agent is also administered to the subject. In embodiments, the therapeutic agent is an agent that treats an inflammatory disease. In embodiments, the therapeutic agent is an agent that treats a chronic inflammatory disease. In embodiments, the therapeutic agent is an agent that treats an autoimmune disease. In embodiments, the therapeutic agent is an agent that treats an infectious disease. In embodiments, the therapeutic agent is an agent that treats cancer.

In embodiments, the poxvirus and/or the stem cell and/or the beta cell are administered to the subject by intravenous, intraperitoneal, intrathecal, intra-cerebro-ventricular, intrapleural, intra-parencymal, intraventricular, intraarticular, or intraocular injection. In embodiments, the poxvirus and/or the stem cell and/or the beta cell are administered directly to a region affected by the disease. In embodiments, the poxvirus and/or the stem cell and/or the beta cell are administered by MRI-guided delivery.

In embodiments, the stem cell and/or the beta cell is autologous to the subject. In embodiments, the stem cell and/or the beta cell is allogeneic to the subject.

In one aspect is provided a method for preserving iPSCs from a patient. In embodiments, the iPSCs are derived from a patient and stored until they are needed to treat a disease. In embodiments, the method includes: (a) obtaining a plurality of somatic cells from a subject; (b) de-differentiating each subset of somatic cells to produce iPSCs; and (c) storing the iPSCs for a period of time. In embodiments, a first subset of somatic cells are obtained from an ectodermal cell type, a second subset of somatic cells are obtained from an endodermal cell type, and a third subset of somatic cells are obtained from a mesodermal cell type. In embodiments, the first, second and third subsets of somatic cells are de-differentiated to produce a first subset of iPSCs, a second subset of iPSCs, and a third subset of iPSCs, respectively.

In embodiments, the iPSCs are stored in a frozen or cryopreserved state. In embodiments, the iPSCs are stored in liquid nitrogen. In embodiments, the iPSCs are stored in the presence of a cryoprotective agent, e.g., glycerol. In embodiments, each subset of iPSCs are stored separately from each other subset of iPSCs. In embodiments, the iPSCs are stored for between one month and 100 years.

In embodiments, a label is associated with each subset of iPSCs. In embodiments, the label identifies the subject.

In embodiments, the iPSCs are stored until the subject is diagnosed with a disease that can be treated by iPSCs. In embodiments, the iPSCs are stored until the subject is diagnosed with a disease that can be treated by iPSCs in combination with a virus. In embodiments, the iPSCs are stored until the subject is diagnosed with a disease that can be treated by iPSCs in combination with a poxvirus. In embodiments, the iPSCs are administered to the subject to treat the disease. In embodiments, the iPSCs and a virus, e.g. a poxvirus, are administered to the subject to treat the disease. In embodiments, the poxvirus is a vaccinia virus.

In embodiments, the iPSCs are genetically modified prior to administration to a subject. For example, the iPSCs may be engineered to express a therapeutic or other relevant molecule, to undergo cell death in response to a stimuli, to increase infectivity by the virus, etc.

In embodiments, a CAR-T cell is also administered to the subject. In embodiments, the CAR targets an antigen associated with the disease. In embodiments, the CAR-T cell is autologous. In embodiments, the CAR-T cell is allogeneic.

In an aspect a method for vaccinating a subject against an infectious disease is provided. The method may include administering to the subject an iPSC and a vaccine. The method may include administering to the subject a beta cell and a vaccine. In embodiments, the disease is smallpox. Without being bound by theory, it is believed that less viral load is needed because iPSCs or beta cells serve as amplification sites. This leads to fewer adverse effects.

In embodiments, immune parameter data and/or other relevant data are collected from the iPSCs from a patient and stored in a system, e.g. the Codex system. Such information can be used for future immunotherapy design of personalized cancer treatments.

In an aspect personalized iPSC banks for tissue and organ therapy are provided. Such banks may contain iPSCs from a plurality of individuals.

In an aspect is provided a method for post-surgery treatment in a patient who had breast cancer surgery. In embodiments, iPSCs or beta cells are administered to an area near or at the site of the surgery, e.g., a breast area. In embodiments, a poxvirus is administered concurrently with the iPSCs or beta cells. In embodiments, administration of the iPSCs or beta cells and optionally poxvirus reduces recurrence of the breast cancer. In embodiments, the iPSCs or beta cells and optionally poxvirus is administered in connection with breast reconstruction.

### BRIEF DESCRIPTION OF THE FIGURES

FIG. 1 shows photographs of iPSCs generated from skin fibroblasts infected with modified vaccinia virus expressing red fluorescent protein (RFP), 8 h (left panel) and 32 h (right panel) after infection at a multiplicity of infection (MOI) of 1.

FIG. 2 shows photographs of iPSCs generated from peripheral blood mononuclear cells (PBMCs) infected with modified vaccinia virus expressing red fluorescent protein (RFP), 8 h (left panel) and 32 h (right panel) after infection at a multiplicity of infection (MOI) of 1.

FIG. 3 shows representations of two genetically modified vaccinia virus strains that are transfected with genes for fluorescent proteins in order to observe infection and viral replication.

FIG. 4 shows infection in cell culture (2D) of Celprogen Human Pancreatic Islets of Langerhans cells. Images were acquired of small segments with 20× magnification using the “IncuCyte” device. Top row (A-E): cells were infected with GLV-1h68 at an MOI of 1. Middle row (F-J): cells were infected with SI-C1-Opt1 at an MOI of 0.1. Bottom row (K-O): cells were infected with SI-C1-Opt1 at an MOI of 1.

FIG. 5 shows a picture of pancreatic islet cells in cell culture, at initial seeding (left panel, A) and after approximately 24 hours (right panel, B).

FIGS. 6A-6D show fluorescence analysis of virus-infected pancreatic islet cells in 2D in vitro culture. FIGS. 6A and 6B: Mean fluorescence intensity of infected pancreatic islet cells measured in counts per well. FIGS. 6C and 6D: Mean fluorescent object area of infected pancreatic islet cells measured in μm²/well.

FIG. 7 shows results of a standard plaque assay of vaccinia virus strains.

### DETAILED DESCRIPTION

After reading this description it will become apparent to one skilled in the art how to implement the invention in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present invention as set forth below.

Before the present invention is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description of the invention is divided into various sections only for the reader's convenience and disclosure found in any section may be combined with that in another section. Titles or subtitles may be used in the specification for the convenience of a reader, which are not intended to influence the scope of the present invention.

### Definitions

Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings:

The terminology used herein is for the purpose of describing particular embodiments only and is not intended to be limiting of the invention. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to a dose amount means that the dose may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

As used herein, the term “concurrently” as referring to administration of a poxvirus and an iPSC, refers to administration within 48 hours of each other. In some embodiments, the poxvirus and cell are administered within 36 hours of each other, within 24 hours of each other, within 12 hours of each other, within 10 hours of each other, within 8 hours of each other, within 6 hours of each other, within 4 hours of each other, within two hours of each other, within 1 hour of each other. In embodiments, the poxvirus and cell are combined prior to administration to the subject.

The term “autologous,” “autologous cell” or “autologous transplantation” as used herein in relation to cell transplantation indicates that the donor and recipient of the cells is the same individual. The term “allogenic,” “allogenic cell” or “allogenic transplantation” as used herein in relation to cell transplantation indicates that the donor and recipient of the cells are different individuals of the same species.

The term “tumor” or “tumor cell,” as used herein, refers to any type of tumor, including solid tumors or non-solid tumors, dispersed tumors, metastatic or disseminated tumors, or tumor cells from any form of tumor.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient,” “subject,” or “subject in need thereof” refers to a living organism suffering from or prone to a disease or condition that can be treated by administration of a pharmaceutical composition as provided herein. Non-limiting examples include humans, other mammals, bovines, rats, mice, dogs, cats, monkeys, goat, sheep, cows, deer, and other non-mammalian animals. In embodiments, a patient is human. In embodiments, the human is a pediatric patient. In embodiments, a patient is a domesticated animal (e.g., goat, sheep, cow, horse, etc.). In embodiments, a patient is a companion animal, including but not limited to canine, feline, rodent (mouse, rat, gerbil, hamster, guinea pig, chinchilla, and the like), rabbit, ferret, etc.

An “effective amount” is an amount sufficient for a compound to accomplish a stated purpose relative to the absence of the compound (e.g. achieve the effect for which it is administered, treat a disease, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As is well known in the art, therapeutically effective amounts for use in humans can also be determined from animal models. For example, a dose for humans can be formulated to achieve a dose that has been found to be effective in animals. The dosage in humans can be adjusted by monitoring effectiveness and adjusting the dosage upwards or downwards, as described herein. Adjusting the dose to achieve maximal efficacy in humans based on the methods described herein and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the composition being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the composition. Thereafter, the dosage is increased by small increments until the optimum effect under circumstances is reached. Dosage amounts and intervals can be adjusted individually to provide levels of the administered composition effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intra-cerebro-ventricular, intrapleural, intra-parencymal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, etc. Administration also includes direct administration, e.g., directly to a site of inflammation. Direct administration may be via guided delivery, e.g., magnetic resonance imaging (MRI)-guided delivery. In embodiments, the administering does not include administration of any active agent other than the recited active agent.

“Co-administer” is meant that a composition described herein is administered at the same time, just prior to, or just after the administration of one or more additional therapies. The compositions provided herein can be administered alone or can be co-administered to the patient. Co-administration is meant to include simultaneous or sequential administration of the compositions individually or in combination (more than one composition). Thus, the preparations can also be combined, when desired, with other active substances.

As used herein, the terms “beta cell,” “pancreatic beta cell,” “beta islet cell” and the like are used interchangeably and refer to cells found in pancreatic islets that synthesize and secrete insulin. Beta cells as described herein may be derived from any source. In some embodiments, the beta cell may be a xenographic beta cell (derived from a species other than the species to be treated, e.g., a non-human source, for example porcine beta cell). Beta cells may be derived by differentiation of stem cells. Differentiation of beta cells is well known in the art, for example as described in WO2000/047720; WO2009/012428; WO2014/160413; and WO2003/026584; each of which is incorporated herein in its entirety for everything taught therein, including all methods, reagents, compositions, and the like.

Variola virus is the cause of smallpox. In contrast to variola virus, vaccinia virus, which has been used for smallpox vaccination, does not normally cause systemic disease in immune-competent individuals and it has therefore been used as a live vaccine to immunize against smallpox. Successful worldwide vaccination with Vaccinia virus culminated in the eradication of smallpox as a natural disease in the 1980s. Since then, vaccination has been discontinued for many years, except for people at higher risk of poxvirus infections (e.g., laboratory workers). Although the United States discontinued routine childhood immunization against smallpox in 1972, the use of smallpox vaccine is generally considered safe for pediatric use.

In some embodiments, an attenuated strain derived from a pathogenic virus is used for the manufacturing of a live vaccine. Non-limiting examples of viral strains that have been used as a smallpox vaccine include but are not limited to the Lister (also known as Elstree), New York City Board of Health (“NYCBH strain”), Dairen, Ikeda, LC16M8, Western Reserve (WR), Copenhagen, Tashkent, Tian Tan, Wyeth, IHD-J, and IHD-W, Brighton, Ankara, MVA, Dairen I, LIPV, LC16MO, LIVP, WR 65-16, EM63, and Connaught strains. In some embodiments, the smallpox vaccine utilized in the methods disclosed herein is an attenuated New York City Board of Health (NYCBOH) strain of vaccinia virus. In some embodiments, the NYCBOH strain of vaccinia virus may be ATCC VR-118 or CJ-MVB-SPX.

In some embodiments, the smallpox vaccine is non-attenuated. In some embodiments, the smallpox vaccine is attenuated.

In some embodiments, the smallpox vaccine is selected from Dryvax, ACAM1000, ACAM2000, Lister, EM63, LIVP, Tian Tan, Copenhagen, Western Reserve, or Modified Vaccinia Ankara (MVA). In some embodiments, the smallpox vaccine is not deficient in any genes present in one or more of these strains.

In some embodiments, the smallpox vaccine is a replication competent virus. In some embodiments, the smallpox vaccine is replication deficient.

The methods and compositions disclosed herein can be used to treat any solid tumor or hematologic malignancy. Tumors that can be treated by the methods disclosed herein include, but are not limited to a bladder tumor, breast tumor, prostate tumor, carcinoma, basal cell carcinoma, biliary tract cancer, bladder cancer, bone cancer, brain cancer, CNS cancer, glioma tumor, cervical cancer, choriocarcinoma, colon and rectum cancer, connective tissue cancer, cancer of the digestive system, endometrial cancer, esophageal cancer, eye cancer, cancer of the head and neck, gastric cancer, intra-epithelial neoplasm, kidney cancer, larynx cancer, leukemia, liver cancer, lung cancer, lymphoma, Hodgkin's lymphoma, Non-Hodgkin's lymphoma, melanoma, myeloma, neuroblastoma, oral cavity cancer, ovarian cancer, pancreatic cancer, retinoblastoma, rhabdomyosarcoma, rectal cancer, renal cancer, cancer of the respiratory system, sarcoma, skin cancer, stomach cancer, testicular cancer, thyroid cancer, uterine cancer, and cancer of the urinary system, such as lymphosarcoma, osteosarcoma, mammary tumors, mastocytoma, brain tumor, melanoma, adenosquamous carcinoma, carcinoid lung tumor, bronchial gland tumor, bronchiolar adenocarcinoma, small cell lung cancer, non-small cell lung cancers, fibroma, myxochondroma, pulmonary sarcoma, neurosarcoma, osteoma, papilloma, retinoblastoma, Ewing's sarcoma, Wilm's tumor, Burkitt's lymphoma, microglioma, neuroblastoma, osteoclastoma, oral neoplasia, fibrosarcoma, osteosarcoma and rhabdomyosarcoma, genital squamous cell carcinoma, transmissible venereal tumor, testicular tumor, seminoma, Sertoli cell tumor, hemangiopericytoma, histiocytoma, chloroma, granulocytic sarcoma, corneal papilloma, corneal squamous cell carcinoma, hemangiosarcoma, pleural mesothelioma, basal cell tumor, thymoma, stomach tumor, adrenal gland carcinoma, oral papillomatosis, hemangioendothelioma, cystadenoma, follicular lymphoma, intestinal lymphosarcoma, fibrosarcoma, and pulmonary squamous cell carcinoma, leukemia, hemangiopericytoma, ocular neoplasia, preputial fibrosarcoma, ulcerative squamous cell carcinoma, preputial carcinoma, connective tissue neoplasia, mastocytoma, hepatocellular carcinoma, lymphoma, pulmonary adenomatosis, pulmonary sarcoma, Rous sarcoma, reticulo-endotheliosis, fibrosarcoma, nephroblastoma, B-cell lymphoma, lymphoid leukosis, retinoblastoma, hepatic neoplasia, lymphosarcoma, plasmacytoid leukemia, swimbladder sarcoma (in fish), caseous lumphadenitis, lung carcinoma, insulinoma, lymphoma, sarcoma, salivary gland tumors, neuroma, pancreatic islet cell tumor, gastric MALT lymphoma and gastric adenocarcinoma.

In some embodiments, the tumor is selected from metastatic melanoma; esophageal and gastric adenocarcinoma; cholangiocarcinoma (any stage); pancreatic adenocarcinoma (any stage); gallbladder cancer (any stage); high-grade mucinous appendix cancer (any stage); high-grade gastrointestinal neuroendocrine cancer (any stage); mesothelioma (any stage); soft tissue sarcoma; prostate cancer; renal cell carcinoma; lung small cell carcinoma; lung non-small cell carcinoma; head and neck squamous cell carcinoma; colorectal cancer; ovarian carcinoma; hepatocellular carcinoma; and glioblastoma.

In some embodiments, the tumor is selected from: glioblastoma, breast carcinoma, lung carcinoma, prostate carcinoma, colon carcinoma, ovarian carcinoma, neuroblastoma, central nervous system tumor, and melanoma.

In some embodiments, the tumor or cancer that can be treated is a childhood or pediatric tumor or cancer. For example, the tumor or cancer can be a leukemia, a lymphoma, a sarcoma, and the like. Non-limiting examples of leukemia include acute lymphoblastic leukemia (ALL) and acute myeloid leukemia (AML). Non-limiting examples of types of lymphomas include Hodgkin disease (or Hodgkin lymphoma) and non-Hodgkin lymphoma (e.g., B and T cell lymphomas). Non-limiting examples of solid tumors or cancers for pediatric patients include brain tumors, Ewing Sarcoma, eye cancer (retinoblastorna), germ cell tumors, Kidney tumors (e.g., Wilms Tumor), liver cancer, neuroblastoma, osteosarcoma, rhabdornyosarcoma, skin cancer (e.g., melanoma), soft tissue sarcoma and thyroid cancer. In some embodiments, the subject is human. In some embodiments, the subject is a pediatric patient. In some embodiments, the subject is a neonate. In some embodiments, the subject is an infant. In some embodiments, the subject is a child. In some embodiments, the subject is an adolescent. In some embodiments, the subject is greater than 12 months in age. In some embodiments the subject is ales than 18 years in age.

In embodiments, the inflammatory disease is enteric fistula, chronic radiation damage (which causes inflammatory tissue defects such as radiation cystitis or radiation enteritis), duodenal ulcers, or a chronic inflammatory disease of the central nervous system, such as post stroke neuro-inflammation, schizophrenia, autism, addiction, chronic traumatic encephalopathy, or vaccine induced neuro-toxicity.

In embodiments, the chronic inflammatory disease is transplant rejection, Dupytren's contracture, peyronies, periodontitis, endometriosis, hepatitis, glomerunephritis, atherscleroisis, cardiovascular disease, arthritis (e.g., osteoarthritis, rheumatoid arthritis, or psoriatic arthritis), inflammatory brain disease (including post-stroke, encephalitis), atherosclerosis, traumatic injury, infection, and/or shock. In an embodiment, the inflammatory disease is Chronic Obstructive Pulmonary Disease (COPD), such as emphysema, chronic bronchitis, or refractory (non-reversible) asthma.

In an embodiment, the autoimmune disease is Myasthenia gravis (MG), Hashimoto's thyroiditis, vasculitis, Graves' disease, psoriasis, Chronic inflammatory demyelinating polyneuropathy (CIDP), Guillain barre, diabetes mellitus type 1, lupus, multiple sclerosis, rheumatoid arthritis, Addison's disease, Sjogren's syndrome, celiac disease, myositis, ankylosing spondylitis, or scleroderma.

In embodiments, the inflammatory disease is enteric fistula, chronic radiation damage (which causes inflammatory tissue defects such as radiation cystitis or radiation enteritis), duodenal ulcers, or a chronic inflammatory disease of the central nervous system, such as post stroke neuro-inflammation, schizophrenia, autism, addiction, chronic traumatic encephalopathy, or vaccine induced neuro-toxicity.

In embodiments, the disease is an infectious disease, traumatic injury, and/or shock.

In embodiments, a chimeric antigen receptor (CAR)-T cell is also administered to the subject. In embodiments, the CAR targets an antigen associated with the disease. In embodiments, the CAR-T cell is autologous. In embodiments, the CAR-T cell is allogeneic. In embodiments, the CAR-T cell and the iPSC are derived from the same individual. In embodiments, the CAR-T cell and the iPSC are derived from different individuals.

Methods of making and using CAR-T cells are well known in the art, for example as disclosed in U.S. Pat. No. 9,328,156, which is incorporated herein by reference in its entirety for all that is taught therein. In embodiments, the CAR-T cells are derived from pluripotent stem cells, for example as described in U.S. Patent Publication No. 2016/0009813, which is incorporated herein by reference in its entirety for all that is taught therein. In embodiments, the CAR-T cells are derived from iPSCs.

In embodiments, the cells to be used for production of the CAR-T cells and the cells to be used for production of the iPSCs (and/or beta cells) are obtained from the same sample, e.g., a blood sample from a subject.

In embodiments, a therapeutic molecule (therapeutic agent) is administered to the subject. Preferably, the therapeutic molecule (therapeutic agent) treats the disease. The therapeutic molecule (therapeutic agent) may be administered as part of the poxvirus/stem cell composition and/or separately. Where the therapeutic molecule (therapeutic agent) is administered as part of the poxvirus/stem cell composition, the therapeutic molecule (therapeutic agent) may be a separate component of the composition. Alternatively (or in addition), the therapeutic molecule may be expressed by the stem cell and/or encoded by the poxvirus.

In embodiments, the therapeutic molecule (therapeutic agent) is selected from abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab (Zinbryta), and muromonab (Orthoclone OKT3).

In embodiments, the therapeutic molecule is an antibiotic. Antibiotics are well known in the art. The antibiotic may be any antibiotic. A skilled clinician can determine what antibiotic should be used based on the type of infection, as well as other standard determinations. Non-limiting examples of antibiotics are actinomycin, bacitracin, colistin, polymyxin B, gramicidins, polymyxins, bacitracins, glycopeptides, and the like.

Compositions and Methods

Compositions and methods of administering poxvirus, including in combination with stem cells, for treating cancer have been described, for example in U.S. Pub. Nos. 2018/0326048; 2017/0239338; U.S. Pat. Nos. 9,005,602; 8,586,022; 10,105,436; and 10,238,700; each of which is incorporated by reference herein in its entirety.

Immunogenic cell death inducers, like viruses, are subject to significant elimination and/or neutralization following systemic application. Therefore, in some embodiments, disclosed herein are suitable vehicles for shielding the disclosed poxvirus, e.g. smallpox vaccine, from the elements of the humoral and cellular immunity in the blood stream, as well as methods for their targeted delivery to tumor sites.

Thus, in some embodiments, disclosed herein is a method of treating a solid tumor or a hematological malignancy in a subject, comprising administering to the subject a poxvirus, e.g. smallpox vaccine, concurrently with an iPSC. Thus, in some embodiments, disclosed herein is a method of treating a solid tumor or a hematological malignancy in a subject, comprising administering to the subject a poxvirus, e.g. smallpox vaccine, concurrently with a beta cell.

In some embodiments, the poxvirus, e.g. smallpox vaccine or other composition as described herein, and the iPSC or beta cell are administered simultaneously. In some embodiments, the poxvirus, e.g. smallpox vaccine and the iPSC or beta cell are administered simultaneously through one administration vehicle. In some embodiments, the poxvirus, e.g. smallpox vaccine and the iPSC or beta cell are administered simultaneously through one vessel, e.g. a syringe, via intratumoral, intravenous, intraperitoneal, intrathecal, intraventricular, intraarticular, or intraocular injection or intradermal injection, or any suitable methods delivering thereof.

In embodiments, the poxvirus and/or the iPSC and/or beta cell are administered to the subject by intravenous, intraperitoneal, intrathecal, intraventricular, intraarticular, intra-cerebro-ventricular, intrapleural, intra-parencymal, or intraocular injection. In embodiments, the poxvirus and/or iPSC and/or beta cell are administered directly to a region affected by the disease. In embodiments, the poxvirus and/or iPSC and/or beta cell are administered by direct injection. In embodiments, the poxvirus and/or iPSC and/or beta cell are administered by MRI-guided delivery.

In embodiments, CAR-T cells are administered in combination with the poxvirus and iPSC (and/or beta cell). In some embodiments, the poxvirus, iPSC (and/or beta cell) and CAR-T cells are administered simultaneously. In some embodiments, the poxvirus, iPSC (and/or beta cell) and CAR-T cells are administered simultaneously through one administration vehicle. In some embodiments, the poxvirus and iPSC (and/or beta cell) are administered separately from the CAR-T cells, e.g., using a different vessel, at a different time, using a different administration method, etc. Methods for administering CAR-T cells are well known in the art, and can be determined by a skilled clinician.

In some embodiments, the compositions disclosed herein comprise a pharmaceutically acceptable carrier. As used herein, the term “pharmaceutically acceptable carrier” refers to solvents, diluents, preservatives, dispersion or suspension aids, isotonic agents, thickening or emulsifying agents, solid binders, and lubricants, appropriate for the particular dosage form. The skilled artisan is aware of a variety of different carriers that may be used in formulating pharmaceutical compositions and knows techniques for the preparation thereof (See Remington's Pharmaceutical Sciences Ed. by Gennaro, Mack Publishing, Easton, Pa., 1995; which is incorporated herein in its entirety by reference). The pharmaceutically acceptable carriers may include, but are not limited to Ringer's solution, isotonic saline, starches, potato starch, sugars, glucose, powdered tragacant, malt, gelatin, talc, cellulose and its derivatives, ethyl cellulose, sodium carboxymethyl cellulose, cellulose acetate excipients, cocoa butter, suppository waxes, agar, alginic acid, oils, cottonseed oil, peanut oil, safflower oil, sesame oil, olive oil, soybean oil, corn oil, glycols, propylene glycol, esters, ethyl laurate, ethyl oleate, buffering agents, aluminum hydroxide, magnesium hydroxide, phosphate buffer solutions, pyrogen-free water, ethyl alcohol, other non-toxic compatible lubricants, sodium lauryl sulfate, magnesium stearate, coloring agents, releasing agents, coating agents, sweetening, flavoring and perfuming agents. Pharmaceutically acceptable carriers may also include preservatives and antioxidants. One or more of the above-mentioned materials can be specifically excluded from the compositions and methods of some embodiments.

A composition disclosed herein comprising a live smallpox vaccine may comprise an adjuvant. Optionally, one or more compounds having adjuvant activity may be included in the vaccine. Adjuvants are non-specific stimulators of the immune system. They enhance the immune response of the host to the vaccine. Examples of adjuvants known in the art are Freund's Complete and Incomplete adjuvant, vitamin E, non-ionic block polymers, muramyldipeptides, ISCOMs (immune stimulating complexes), saponins, mineral oil, vegetable oil, and Carbopol. Adjuvants, especially suitable for mucosal application are, for example, E. coli heat-labile toxin (LT) or Cholera toxin (CT). Other suitable adjuvants are for example aluminium hydroxide, aluminium phosphate or aluminium oxide, oil-emulsions (e.g., of Bayol F® or Marcol 52®, saponins or vitamin-E solubilisate). One or more of the above-mentioned materials can be specifically excluded from the compositions and methods of some embodiments.

The effective dosage of each of the treatment modalities disclosed herein may vary depending on various factors, including but not limited to the particular treatment, compound or pharmaceutical composition employed, the mode of administration, the condition being treated, and/or the severity of the condition being treated. Thus, the dosage regimen of the combination of the invention is selected in accordance with a variety of factors including the route of administration and the renal and hepatic function of the patient.

A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites.

Methods of preparing pharmaceutical compositions comprising the relevant treatments disclosed herein are known in the art and will be apparent from the art, from known standard references, such as Remington's Pharmaceutical Sciences, Mack Publishing Company, Easton, Pa., 18th edition (1990), which is incorporated herein by reference in its entirety.

The amount of vaccine administered to an average-sized adult can be, for example, 1×10² to 1×10¹⁰ plaque-forming units, 1×10³ to 1×10⁸ plaque-forming units 1×10⁴ to 1×10⁶ plaque-forming units, or any value or sub range there between. As a specific example, about 2.5×10⁵ plaque-forming units can be used.

It should be understood that the embodiments described herein are not limited to vaccinations or vaccinating per se, but also relate to generating an immune response or reaction to cancer cells. While the words “vaccine,” “vaccination,” or other like terms are used for convenience, it should be understood that such embodiments also relate to immune compositions, immunogenic compositions, immune response generation, immunization, etc., where absolute prophylactic immunity is not required or generated. For example, the embodiments referring to vaccination also can relate to generating or to assisting in creating an immunogenic or immune response against an appropriate antigen (e.g., a tumor cell or tumor), regardless of whether that response results in absolute eradication or immunization against the tumor cell, tumor, cancer, infectious agent, etc.

De-differentiation of somatic cells, e.g. fibroblast cells, to iPSCs is well known. For example, one or more of Oct4, Sox2, Klf4, and c-myc genes may be introduced into the somatic cells. See, e.g., Malik N, Rao MS. A review of the methods for human iPSC derivation. Methods Mol Biol. 2013; 997:23-33. doi:10.1007/978-1-62703-348-0_3; Gonzales et al., Nat Rev Genet. 2011 April; 12(4):231-42. doi: 10.1038/nrg2937. Epub 2011 Feb. 22.; U.S. Pat. Nos 9,580,689; 9,499,797; each of which is incorporated herein by reference in its entirety.

Methods for Producing and Preserving iPSCs

As treatment of disease becomes more personalized, it will be desirable to have autologous cells from a patient in order to treat that patient. However, isolation/harvesting of cells from a patient can be a difficult or painful process. It is therefore desirable to isolate/harvest cells as few times as possible. It may also be desired to isolate/harvest cells before the individual gets sick, and to preserve the cells for later therapies.

Thus, in one aspect is provided a method for preserving iPSCs from a patient. In embodiments, the iPSCs are derived from a patient and stored until they are needed to treat a disease. In embodiments, the method includes: (a) obtaining a plurality of somatic cells from a subject; (b) de-differentiating each subset of somatic cells to produce iPSCs; and (c) storing the iPSCs for a period of time. In embodiments, a first subset of somatic cells are obtained from an ectodermal cell type, a second subset of somatic cells are obtained from an endodermal cell type, and a third subset of somatic cells are obtained from a mesodermal cell type. In embodiments, the first, second and third subsets of somatic cells are de-differentiated to produce a first subset of iPSCs, a second subset of iPSCs, and a third subset of iPSCs, respectively.

In embodiments, the iPSCs are stored in a frozen or cryopreserved state. In embodiments, the iPSCs are stored in liquid nitrogen. In embodiments, the iPSCs are stored in the presence of a cryoprotective agent, e.g., glycerol. In embodiments, each subset of iPSCs are stored separately from each other subset of iPSCs. In embodiments, the iPSCs are stored for between one month and 100 years.

In embodiments, a label is associated with each subset of iPSCs. In embodiments, the label identifies the subject. The label may be any label that can be used to identify the iPSCs, such as a written/typed label, a barcode, a QR code, and the like.

In embodiments, the iPSCs are stored until the subject is diagnosed with a disease that can be treated by iPSCs. In embodiments, the iPSCs are stored until the subject is diagnosed with a disease that can be treated by iPSCs in combination with a virus. In embodiments, the iPSCs are stored until the subject is diagnosed with a disease that can be treated by iPSCs in combination with a poxvirus. In embodiments, the iPSCs are administered to the subject to treat the disease. In embodiments, the iPSCs and a virus, e.g. a poxvirus, are administered to the subject to treat the disease. In embodiments, the poxvirus is a vaccinia virus.

In embodiments, the iPSCs are genetically modified prior to administration to a subject. For example, the iPSCs may be engineered to express a therapeutic or other relevant molecule, to undergo cell death in response to a stimuli, to increase infectivity by the virus, etc.

In embodiments, the iPSCs are differentiated prior to administration to a subject.

In embodiments, the iPSCs may be used as allogenic cells to treat someone other than the person from whom they were derived.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

One skilled in the art would understand that descriptions of making and using the particles described herein is for the sole purpose of illustration, and that the present disclosure is not limited by this illustration.

### Example 1. Infection of iPSCs with Vaccinia Virus

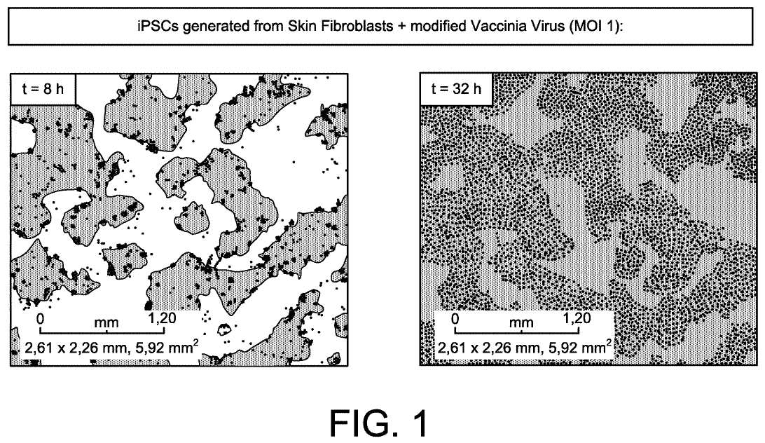

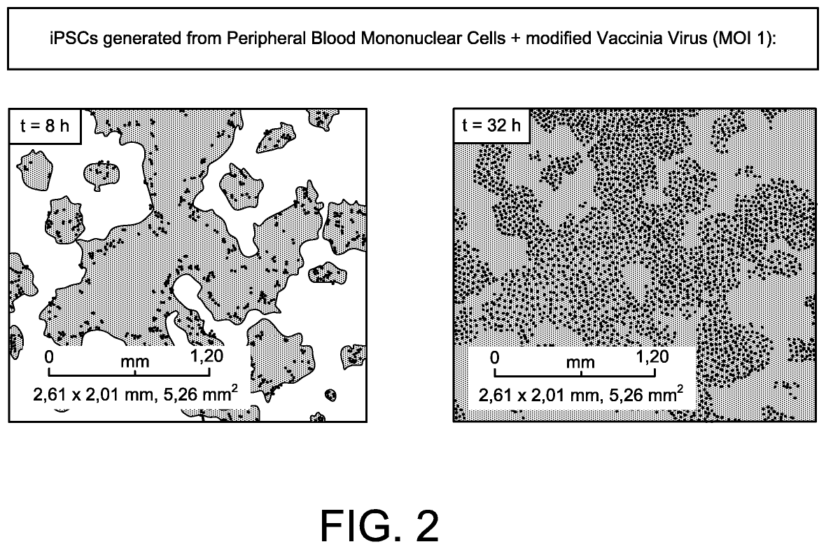

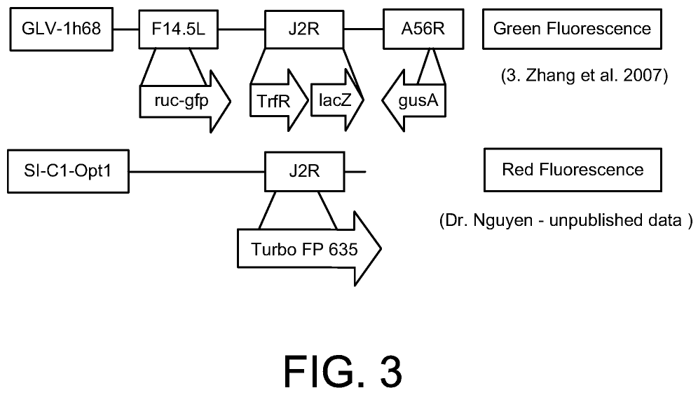

Two iPSC lines (SCVI 20,iPSC, origin human skin fibroblasts; and SCVI15 iPSC origin human peripheral blood mononuclear cells, both from Professor J WU Stanford Univ. USA) were infected with a RFP-expressing vaccinia virus strain at MOI=1. RFP expression from the cells at 8 h and 32 h is shown in FIGS. 1 and 2.

The virus is efficiently taken up, replicates, and causes 95% oncolysis within 3 days after infection. These findings strongly indicate that autologous iPSCs will function as efficient vaccine delivery and short term protection system for Vaccinia constructs from the recipient's innate immune system.

### Example 2

Pancreatic cancer (7th) and liver cancer (5th) are among the leading causes of cancer-associated death. Both cancer types respond poorly to non-surgical approaches, resulting in an urgent need for new therapeutic strategies. Previous research approaches verified that attenuated oncolytic strains of Vaccinia virus (VV) are capable of infection and lysis of human pancreatic cancer (1) and liver cancer cells (2) in vitro and in nude mice xenografts. To avoid viral clearance by the recipient's immune response, we intend to infect certain cell types in order to hide the therapeutic virus until it reaches the site of tumor growth by using those cells as a ‘Trojan horse’. Selected candidates for a Trojan horse system include pancreatic islets of Langerhans. Transplantation of human pancreatic islets in the hepatic artery of diabetic patients is an established clinical approach. The procedure is minimally invasive and normally not associated with major risks. Thus, these cells could be potential carriers for Vaccinia to infect liver cancer and liver metastases of pancreatic cancer upon implantation. We set out to investigate, whether a commercially available human islet cell line is susceptible towards infection with two fluorescent oncolytic VV strains. Experiments revealed that both virus strains successfully infected the pancreatic islet cells but amplified at different extent. For the red VV strain, we observed an 11 fold higher maximum fluorescence intensity and an approximately 18 fold larger area of fluorescence emitting cells than for the green VV strain. The red virus strain infects the islet cells more efficiently and might deliver a larger dose of virus to tumors.

For infection experiments, we used two genetically modified VV strains, GLV-1h68 and SI-C1-Opt1, which are transfected with genes for fluorescent proteins in order to observe infection and viral replication (FIG. 2).

Pancreatic islet cells (stable cell line purchased from “Celprogen—Stem Cell Research and Therapeutics”) were infected in “Advanced RPMI 1640 Medium”, supplemented with 2%/10% FCS and 1% L-Glutamine. Microscopy and measurement of fluorescence over time after virus infection were carried out with the “IncuCyte-Live Cell Analysis” system (Essen BioScience/Sartorious).

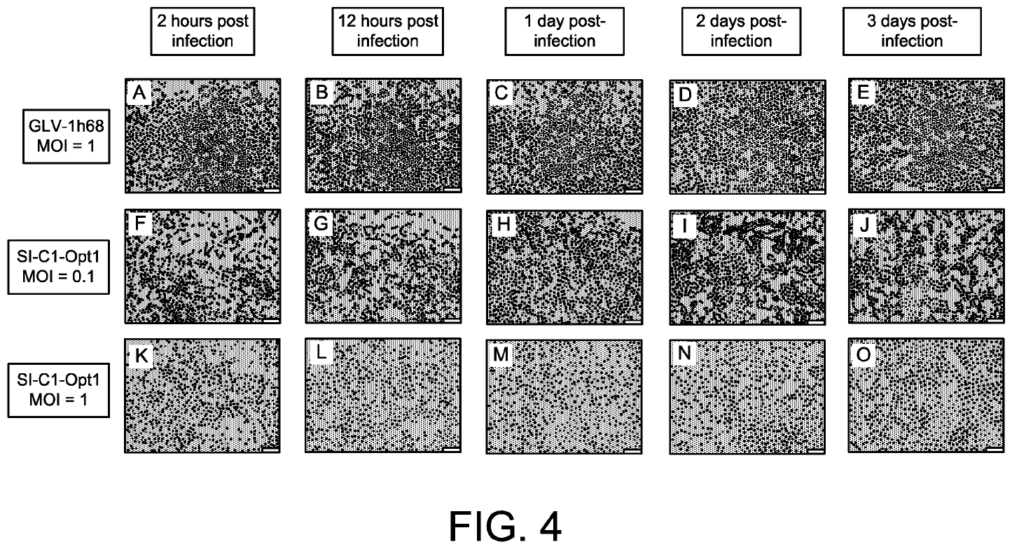

SI-C1-Opt1 Virus infects islet cells more efficiently than GLV-1h68 and spreads to adjacent cells. As shown in FIG. 4 (top row, A-E), in cells infected with GLV-1h68 at an MOI of 1, early fluorescent signals appear 12 hours post infection only in single cells scattered throughout the cell lawn. At 3 days post-infection the fluorescent signals visibly start to fade away. In cells that were infected with the SI-C1-Opt1 at an MOI of 0.1 (middle row, F-J), two hours post-infection single cells show fluorescent signals at low intensity. After 12 hours the intensity of red fluorescence notably increases. During the subsequent days the number of isolated cells declines but at several regions fluorescent signals appear in neighboring cells, implying that the virus spreads to adjacent cells. In cells that were infected with the SI-C1-Opt1 at an MOI of 0.1 (bottom row, K-O), two hours post-infection single cells show fluorescent signals at low intensity. After 12 hours the intensity of red fluorescence notably increases. During the subsequent days the number of isolated cells declines but at several regions fluorescent signals appear in neighboring cells, implying that the virus spreads to adjacent cells.

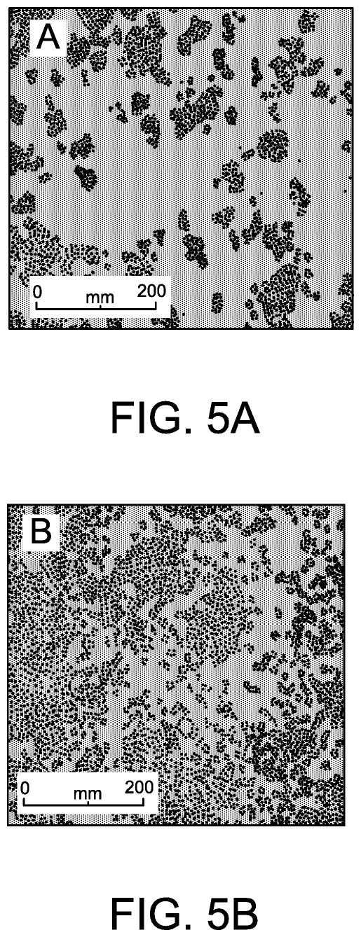

Pancreatic islet cell line exhibits fast proliferation and tends to grow in a monolayer. FIG. 5 shows pancreatic islet cells in culture. Cells were seeded in “Geltrex” coated 24 well plates at a concentration of 5×10⁴ cells/well. Images were acquired of small segments with 20× magnification using the “IncuCyte” device. At initial seeding density of 5×10⁴ cells/well, cells tend to form small cellular clusters (left panel, A). After a growth period of 24 hours, individual clusters start to combine and form a layer (right panel, B).

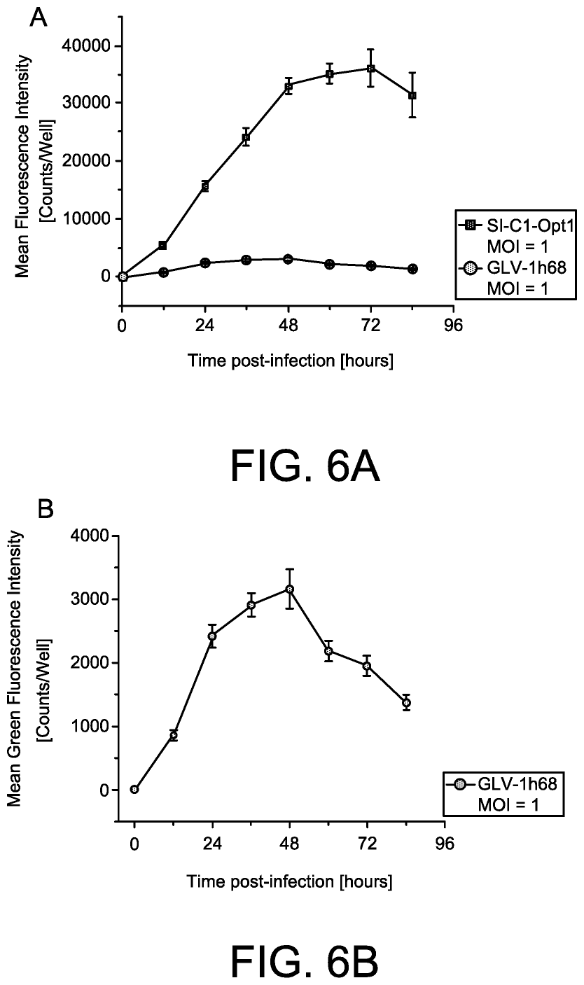

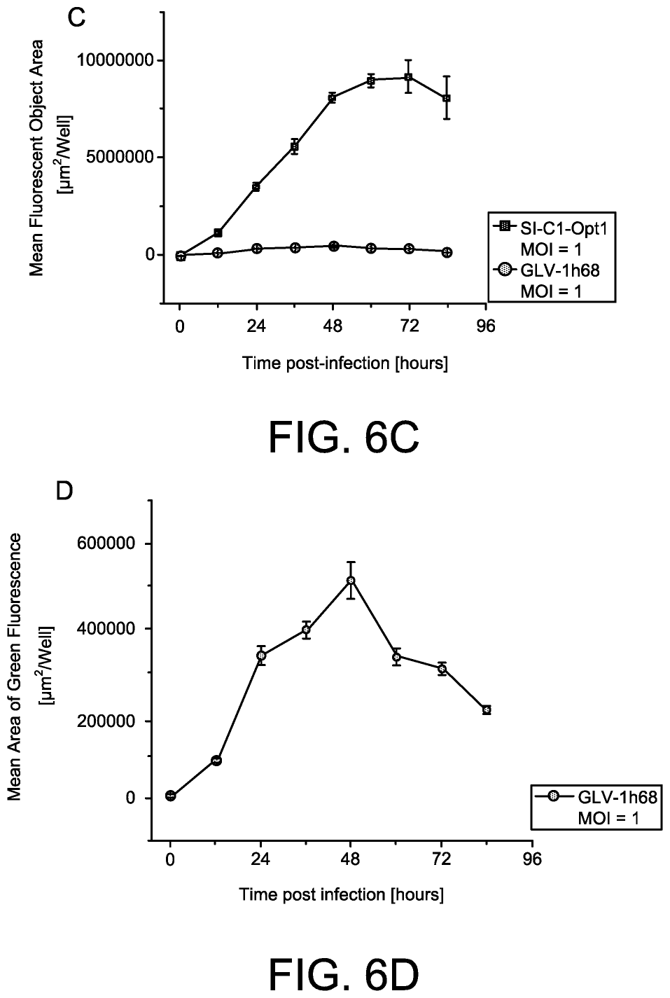

SI-C1-Opt1 displays more than a 10-fold higher viral replication ratio, than GLV-1h68. Cells were infected with an MOI=1 of either GLV1h68 or SI-C1-Opt1. Measurement of fluorescence was carried out by using the “whole well analysis” function of the “IncuCyte” to observe all cells per well at once at 4× magnification. As shown in FIG. 6A and B, infection with the green virus GLV-1h68 reached its maximum signal intensity at 48 hours, followed by a rapid decline, while infection with SI-C1-Opt1 led to an increase of fluorescent counts until 72 hours post-infection. Infection with the red virus SI-C1-Opt1 led to an approximately 11 fold higher maximum mean fluorescence intensity than the green virus GLV-1h68. As shown in FIGS. 6C and 6D, the trends for mean area of fluorescent objects are almost identical to the fluorescence intensity. Infection with the red virus SI-C1-Opt1 led to an 18 fold higher maximum mean area of fluorescence than the green virus.

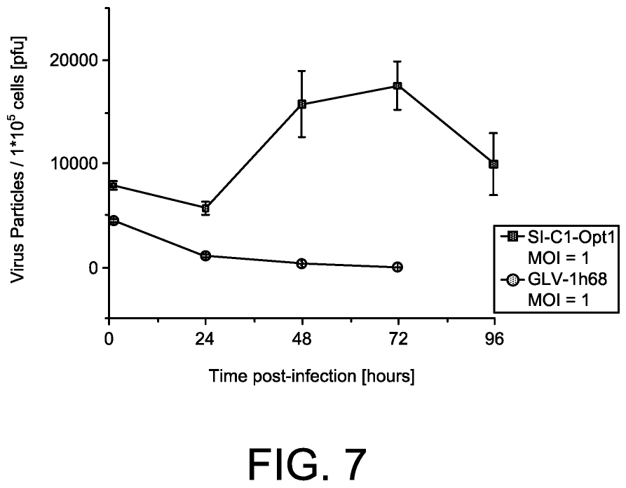

SI-C1-Opt1 titer increases during infection of islet cells, until 72 hours post-infection, GLV-1h68 titer decreases. Pancreatic islet cells were infected with MOI=1 of SI-C1-Opt1/GLV-1h68. Cells were harvested and counted lh post infection and every 24 hours after. Virus particles were released and applied on a CV-1 cellular monolayer to measure the amount of virus particles per 1×10⁵ islet cells. For SI-C1-Opt1 the number of virus particles per cell amount decreases first, due to fast cell proliferation (FIG. 7). After 24 hours the titer increases to its maximum at 72 hours. During infection with GLV-1h68 the number of virus particles gradually decreases over time.

### REFERENCES

1. Dai et al (2014). Oncolytic vaccinia virus in combination with radiation shows synergistic antitumor efficacy in pancreatic cancer. Cancer Letters, 344(2), 282-290.
2. Ady et al (2014). Oncolytic immunotherapy using recombinant vaccinia virus GLV-1h68 kills sorafenib-resistant hepatocellular carcinoma efficiently. Surgery, 156(2), 263-269.
3. Zhang et al (2007). Eradication of solid human breast tumors in nude mice with an intravenously injected light-emitting oncolytic vaccinia virus. Cancer Research, 67(20), 10038-10046.

What is claimed is:

1. A composition comprising a poxvirus and an induced pluripotent stem cell (iPSC).

2. The composition of claim 1, wherein the iPSC is derived from an ectodermal cell type, an endodermal cell type, or a mesodermal cell type.

3. The composition of claim 1 or 2, wherein the iPSC is derived from a subject to be treated with the composition.

4. A composition comprising a poxvirus and a pancreatic beta cell.

5. The composition of claim 4, wherein the pancreatic beta cell is derived from a stem cell.

6. The composition of claim 5, wherein the stem cell is an induced pluripotent stem cell (iPSC).

7. The composition of any one of the above claims, wherein the poxvirus is a vaccinia virus.

8. The composition of claim 7, wherein the vaccinia virus is selected from Dryvax, ACAM1000, ACAM2000, Lister, EM63, LIVP, Tian Tan, Copenhagen, Western Reserve, Modified Vaccinia Ankara (MVA), New York City Board of Health, Dairen, Ikeda, LC16M8, Western Reserve Copenhagen, Tashkent, Tian Tan, Wyeth, IHD-J, and IHD-W, Brighton, Dairen I and Connaught strains.

9. The composition of claim 8, wherein the vaccinia virus is ACAM1000 or ACAM2000.

10. The composition of claim 8, wherein the vaccinia virus is a New York City Board of Health strain.

11. The composition of any one of the above claims, wherein the poxvirus is an attenuated virus.

12. The composition of any one of claims 1-11, wherein the cell comprises a recombinant polynucleotide, wherein said recombinant polynucleotide encodes a therapeutic molecule.

13. The composition of any one of claims 1-12, wherein the poxvirus comprises a recombinant polynucleotide, wherein said recombinant polynucleotide encodes a therapeutic molecule.

14. The composition of any one of claims 1-13, further comprising a chimeric antigen receptor (CAR)-T cell.

15. The composition of claim 14, wherein the CAR-T cell and the iPSC or beta cell were derived from the same individual.

16. The composition of claim 14 or 15, wherein the CAR-T cell and/or the iPSC and/or the beta cell were derived from a patient to be treated with the therapeutic composition.

17. The composition of any one of claims 14 to 16, wherein the CAR targets an antigen associated with a disease.

18. The composition of claim 17, wherein the disease is cancer, an inflammatory disease, or an infectious disease.

19. A pharmaceutical composition comprising the composition of any one of claims 1 to 18 and a pharmaceutically acceptable excipient.

20. A method for treating a disease in a subject, the method comprising administering to the subject a composition of any one of claims 1 to 19.

21. A method for treating a disease characterized by chronic inflammation in a subject in need thereof, the method comprising administering to the subject a composition of any one of claims 1 to 19.

22. The method of claim 21, wherein the chronic inflammatory disease is selected from asthma, chronic peptic ulcer, tuberculosis, arthritis, periodontitis, ulcerative colitis, Crohn's disease, sinusitis, active hepatitis, atherosclerosis, dermatitis, inflammatory bowel disease (IBS), systemic lupus, fibromyalgia, Type 1 diabetes, psoriasis, Multiple sclerosis, Addison's disease, Grave's disease, Sjogren's syndrome, Hashimoto's thyroiditis, Myasthenia gravis, vasculitis, pernicious anemia, or celiac disease.

23. A method for treating cancer in a subject in need thereof, the method comprising administering to the subject a composition of any one of claims 1 to 19.

24. The method of any one of claims 20 to 23, further comprising administering a therapeutic agent to the subject.

25. The method of claim 24, wherein the therapeutic agent is an agent that treats the disease.

26. The method of any one of claims 20 to 25, wherein the poxvirus and/or the stem cell are administered to the subject by intravenous, intraperitoneal, intrathecal, intra-cerebro-ventricular, intrapleural, intra-parencymal, intraventricular, intraarticular, or intraocular injection.

27. The method of any one of claims 20 to 25, wherein the poxvirus and/or the cell are administered directly to a region affected by the disease.

28. The method of any one of claims 20 to 25, wherein the poxvirus and/or the stem cell or beta cell are administered by Mill-guided delivery.

29. The method of any one of claims 20 to 28, wherein the stem cell or beta cell is autologous to the subject.

30. The method of any one of claims 20 to 29, wherein the T cell is autologous to the subject.

31. The method of any one of claims 20 to 28, wherein the stem cell or beta cell is allogeneic to the subject.

32. The method of any one of claim 20 to 29 or 31, wherein the T cell is allogeneic to the subject.

33. A method for preserving induced pluripotent stem cells (iPSCs) from a subject, the method comprising:
(a) obtaining a plurality of somatic cells from the subject, wherein a first subset of somatic cells are obtained from an ectodermal cell type, a second subset of somatic cells are obtained from an endodermal cell type, and a third subset of somatic cells are obtained from a mesodermal cell type;
(b) de-differentiating each subset of somatic cells to produce a first subset of iPSCs, a second subset of iPSCs, and a third subset of iPSCs; and
(c) storing each subset of iPSCs for a period of time.

34. The method of claim 33, wherein the iPSCs are stored in a frozen or cryopreserved state.

35. The method of claim 33 or 34, wherein each subset of iPSCs are stored separately from each other subset of iPSCs.

36. The method of any one of claims 33 to 35, further comprising associating a label with each subset of iPSCs, wherein the label identifies the subject.

37. A method for treating a subject having a disease, the method comprising administering to a subject at least one subset of iPSCs made by the method of any one of claims 33 to 36 and a poxvirus.

38. The method of claim 37, wherein the poxvirus is a vaccinia virus.

39. The method of claim 38, wherein the vaccinia virus is selected from Dryvax, ACAM1000, ACAM2000, Lister, EM63, LIVP, Tian Tan, Copenhagen, Western Reserve, Modified Vaccinia Ankara (MVA), New York City Board of Health, Dairen, Ikeda, LC16M8, Western Reserve Copenhagen, Tashkent, Tian Tan, Wyeth, IHD-J, and IHD-W, Brighton, Dairen I and Connaught strains.

40. The method of claim 39, wherein the vaccinia virus is ACAM1000 or ACAM2000.

41. The method of claim 39, wherein the vaccinia virus is a New York City Board of Health strain.

42. The method of any one of claims 37 to 41, wherein the poxvirus is an attenuated virus.

43. The method of any one of claims 37 to 42, wherein the stem cell comprises a recombinant polynucleotide, wherein said recombinant polynucleotide encodes a therapeutic molecule.

44. The method of any one of claims 37 to 43, wherein the poxvirus comprises a recombinant polynucleotide, wherein said recombinant polynucleotide encodes a therapeutic molecule.

45. The method of any one of claims 37 to 44, further comprising administering a CAR-T cell to the subject.

46. The method of claim 45, wherein the CAR targets an antigen associated with the disease.

47. The method of claim 45 or 46, wherein the T cell is autologous to the subject.

48. The method of claim 45 or 46, wherein the T cell is allogeneic to the subject.

49. The method of any one of claims 37 to 44, wherein the disease is an autoimmune disease.

50. The method of any one of claims 37 to 44, wherein the disease is a cancer.

51. The method of any one of claims 37 to 44, wherein the disease is an inflammatory disease.

52. The method of any one of claims 37 to 44, wherein the disease is an infectious disease.

53. The method of any one of claims 20 to 52, wherein the subject is a human.

54. The method of any one of claims 20 to 52, wherein the subject is a domesticated animal.

55. The method of any one of claims 20 to 52, wherein the subject is a companion animal.

56. The method of claim 55, wherein the subject is a canine.

57. The method of any one of claims 37 to 56, wherein the iPSCs are differentiated prior to administration.

58. The method of claim 57, wherein the iPSCs are differentiated to pancreatic beta cells.
